(19) United States
(12) Patent Application Publication
(10) Pub. No.: US 2010/0324515 A1
(43) Pub. Date: Dec. 23, 2010

(54) PRE-CONSOLIDATED SPUNBONDED WEB, COMPOSITE NONWOVEN COMPRISING SAID PRE-CONSOLIDATED SPUNBONDED WEB, METHOD AND CONTINUOUS SYSTEM FOR PRODUCING SAID COMPOSITE

(75) Inventor: Galliano Boscolo, Vigilano B.se (IT)

Correspondence Address: HARNESS, DICKEY & PIERCE, P.L.C., P.O. BOX 828, BLOOMFIELD HILLS, MI, 48303, US

(73) Assignee: ALBIS SPA, Milano (IT)

(21) Appl. No.: 12/528,287

(22) PCT Filed: Feb. 18, 2008
(86) PCT No.: PCT/EP2008/001236

§ 371 Date: Aug. 21, 2009

(30) Foreign Application Priority Data
Feb. 22, 2007 (EP) 07 003 643.9

(51) Int. Cl. D04H 3/16; A61F 13/15; D04H 3/08

(52) U.S. Cl. 604/367; 442/401; 428/219; 156/181; 156/148; 156/436

(57) ABSTRACT

The pre-consolidated spunbonded web (A) comprises continuous microfilaments having a diameter (D1) less or equal to 15 μm, and bonding dots having a density higher than or equal to 90 dots/cm², and more preferably higher than or equal to 100 dots/cm². This pre-consolidated spunbonded web (A) is used preferably for making composite nonwoven, more especially hydroentangled composite nonwoven (AJBfC) comprising said pre-consolidated spunbonded web (A), and an absorbent pulp layer (P), and a carded or spunbonded cover layer (C).

### FIELD OF THE INVENTION

The present invention relates to a novel pre-consolidated spunbonded web, and to a composite nonwoven comprising several layers, one of said layer being the said pre-consolidated spunbonded web. The invention also relates to a method and continuous system for producing said novel absorbent composite nonwoven. One first preferred application of the invention is the manufacturing of hydroentangled composite nonwoven for absorbing liquids and comprising at least a pre-consolidated spunbonded layer and an absorbent pulp layer. One second preferred application of the invention is the manufacturing of hydroentangled composite nonwoven comprising at least a pre-consolidated spunbonded layer and a carded layer.

### PRIOR ART

### Spunbonding

Spunbonding is a well-know technology used in the field of nonwoven. A Spunbonded web is produced by depositing extruded spun filaments onto a collecting belt in a uniform random manner. The spunbonded web is then pre-consolidated for example by thermo-bonding, i.e. by applying heat and pressure by means of heated rolls. The thermo-bonding partially melt and fuse the filaments together, and imparts strength and integrity to the web.

Pre-consolidated spunbonded webs are widely used in many types of composite nonwoven, and for example in SS, SSS, SMS, SPC, SPS, SC, SMC nonwoven [S: Spunbonded layer; P: Pulp Layer; C: Carded Layer; M: Meltblown layer].

Among these composite nonwovens, SPC or SPS nonwovens are more especially used for absorbing liquids, in particular, but non only, in the hygienic industry. SC nonwovens are also used for making, for example, top sheets of diapers and training pants. In particular, the properties of the spunbonded layers influence the strike trough time of the top sheet.

### Absorbent Composite Nonwoven

Absorbent composite nonwoven are widely used in the prior art for absorbing liquids, especially, but not only, in the hygienic industry for making products such as, for example, diapers or sanitary napkins.

Absorbent composite nonwovens generally comprise at least two layers: a consolidated nonwoven carrier and an absorbent layer.

An absorbent material widely used for making the absorbent layer is a fibre material generally referred as “pulp”, and made of or containing fibres from natural sources such as woody and non-woody plants. Woody plants include, for example, deciduous and coniferous trees. Non-woody plants include, for example, cotton, flax, esparto grass, milkweed, straw, jute hemp, and bagasse.

In European patent application EP 0 540 041, a process for making a hydroentangled absorbent composite nonwoven is being disclosed. Said process comprises the steps of:
providing a nonwoven carrier,
hydraulically needling the nonwoven carrier, in order mainly to enhance the liquid distribution properties of the nonwoven carrier,
applying and bonding a layer of absorbent material, including pulp, onto the surface of the nonwoven carrier.

It has become apparent that pure consolidation of the composite absorbent nonwoven by compression only produces an insufficiently secure contact between the absorbent layer and the carrier nonwoven.

A satisfactory connection of an absorbent pulp layer to a nonwoven carrier is known, e.g. from U.S. Pat. No. 3,560,326 or PCT application WO92/080834, specifically through hydraulic needling of the pulp fibres of the absorbent layer with the consolidated nonwoven carrier.

As pointed out in U.S. Pat. No. 6,836,937, the hydraulic needling of the pulp fibres of the absorbent layer with the consolidated nonwoven carrier results however in a high loss of pulp fibres. According to U.S. Pat. No. 6,836,937, tests have shown that up to 12% of the pulp fibres are washed out of the useful absorbent layer or bond, and are thus lost for the efficiency of the product. Moreover, in theses processes, very many lost pulp fibres get into the filtration means that are necessary for treating and recycling water, in case of water needling. This increases the costs for the water recycling process, and thereby the manufacturing costs of the absorbent composite nonwoven.

U.S. Pat. No. 6,836,937 discloses a new process that overcomes this problem of high pulp loss. This process consists essentially in inserting a thin intermediate meltblown layer between the nonwoven carrier and the absorbent pulp layer. This technical solution increases however the production costs, since it involves the manufacture of a supplementary layer between the nonwoven carrier and the absorbent pulp layer.

PCT applications WO 2004/092472 and WO 2006/010766 disclose a method for manufacturing a hydroentangled composite nonwoven comprising a spunbonded layer and a pulp layer. More particularly, it is recommended in these publications to spin splittable multicomponent polymers filaments for making the spunbonded layer. These splittable multicomponent polymers filaments are composed of microfilaments having a count between 0.1 dtex and 0.9 dtex, and the splittable filaments have a count between 1.7 dtex and 2.2 dtex. The splitting of the filaments is obtained during the hydroentanglement step of the spunbonded layer.

PCT application WO 01/53588 and U.S. Pat. No. 6,836,938 disclose a method for producing a composite nonwoven, in particular for the production of a hygienic product, said method comprising the following steps:
forming a spunbonded nonwoven layer,
compressing and optionally thermo-bonding the spunbonding layer in order to obtain a light bonding of the fibres of the spunbonded layer,
coating the pre-consolidated spunbonded layer with a layer of pulp fibres,
conducting a hydrodynamic water needling process, in order to interconnect and strengthen the layer of pulp fibres and the pre-consolidated spunbonded layer.

One objective of this publication is to reduce the pulp loss during hydrodynamic water needling, and for achieving this objective, it is recommended in this publication to make only a light bonding of the fibres of the spunbonded layer during the compaction and optional thermo-bonding step, in such a way that the pulp fibres enter into an internal bonding with the fibres of the spunbonded nonwoven fabric in the hydrodynamic water needling.

Publication US 2003/0207636 and PCT application WO01/53590 disclose a hydroentangled and absorbent composite nonwoven comprising a bulk pulp layer and a spunbonded layer made of fine denier filaments, typically in the range of 0.5 denier and 1.2 denier (i.e. 0.55 dtex and 1.3 dtex). In this publication, it is pointed out that the type of bonding of the spunbonded layer is not believed to be critical and may include, for example, solvent adhesive, needling, hydroentanglement, or thermal bonding.

### OBJECTIVES OF THE INVENTION

An objective of the invention is to propose a novel pre-consolidated spunbonded web, more especially, but not exclusively, a novel pre-consolidated spunbonded web that is suitable for making hydroentangled composite nonwovens, and more particularly SPC, SPS, or SC nonwovens.

Another objective of the invention is to propose a novel pre-consolidated spunbonded web having a low air permeability and good mechanical properties, in particular good tensile properties.

Another objective of the invention is to propose a novel hydroentangled absorbent composite nonwoven comprising at least a spunbonded layer and an absorbent pulp layer for absorbing liquids, and a process and continuous system for producing said novel composite nonwoven.

Another objective of the invention is to obtain a hydroentangled absorbent composite nonwoven exhibiting improved mechanical properties, in particular good tensile properties, and good abrasion resistance properties.

Another objective of the invention is to obtain a hydroentangled absorbent composite nonwoven exhibiting an improved softness.

Another objective of the invention is to reduce pulp losses during the manufacturing process of the composite, especially during the hydrodynamic water needling step of the layers of the composite.

### SUMMARY OF THE INVENTION

A first object of the invention is thus a method of producing a pre-consolidated spunbonded web (A), as defined in independent claim 1. Said method comprises the steps of:
(a) forming one spunbonded layer (A′),
(b) thermo-bonding the said spunbonded layer (A′), in order to obtain a pre-consolidated spunbonded web (A).

According to the invention the spunbonded layer (A′) formed in step (a) comprises continuous microfilaments having a diameter (DI) less than or equal to 15 μm, and in that the pre-consolidation step (b) of the spunbonded layer (A′) is performed by means of a bonding pattern having bonding dots, the density (DD) of said bonding dots being higher than or equal to 90 dots/cm².

A second object of the invention is a method of producing a hydroentangled composite nonwoven comprising at least two superposed layers, and as defined in independent claim 11. Said method comprises the following steps:
forming one pre-consolidated spunbonded layer (A) according to first above method [steps (a) and (b)],
laying at least one second layer onto said pre-consolidated spunbonded layer (A) [step (c)],
consolidating the layers by hydrodynamic needling [step(d)].

In one variant, the second layer can be a carded layer or a spunbonded layer.

In another variant, the second layer is a pulp layer, in order to obtain a hydroentangled absorbent composite nonwoven. In this variant, the spunbonded layer (A), that comprises very fine continuous microfilaments (DI≦15 μm) and is pre-consolidated with a microbonding pattern having a very high bonding dots density (≧90 dots/cm²), advantageously constitutes a good barrier for the pulp fibres during the hydrodynamic needling of the composite, thereby reducing the pulp loss, and enables to achieve good mechanical properties, good uniformity, and good softness for the composite nonwoven.

Preferably, but not necessarily, all steps (a), (b), (c) and (d) of the method of the invention are advantageously performed continuously on one production line. But within the scope of the invention, some steps of the method can be performed separately on separate production lines. For example, the pre-consolidated spunbonded layer (A) can be produced on a first production line (steps (a) and (b)), and stored in the form of a roll. Then this pre-consolidated spunbonded layer (A) is transported to a second production line, where it can be used for performing the following steps (c) and (d) of the method of the invention. In a similar way, when the method of the invention comprises an additional step of providing a nonwoven cover layer (C) onto and in contact with the absorbent pulp layer (B) before the step (d) of consolidating the composite nonwoven, the said nonwoven cover (C) can be either produced continuously in line with the other steps on the same production line, or can be produced separately on a first production line, and be transported and used on a second production line where the other steps are performed.

The terms “pulp layer” used therein and in the claims encompass any absorbent layer essentially comprising pulp.

The term “pulp” as used therein and in the claims refers to absorbent material made of or containing fibres from natural sources such a as woody and non-woody plants. Woody plants include, for example, deciduous and coniferous trees. Non-woody plants include, for example, cotton, flax, esparto grass, milkweed, straw, jute hemp, and bagasse. Within the scope of the invention, the absorbent pulp layer can be made solely of pulp fibres, but can be also be made of a dry mixture of pulp fibres with other materials, provided the said mixture can be dry-laid onto the consolidated spunbonded layer of the invention, by air-laid techniques or the like.

In a particular variant, the method of the invention further comprises an additional step of providing a nonwoven cover layer (C) onto and in contact with the absorbent pulp layer (B) before the step (d) of consolidating the composite nonwoven. More preferably, this additional step comprises the following sub-steps:
(a′) forming one spunbonded layer (C′)
(b′) thermo-bonding the said spunbonded layer (C′), in order to obtain a pre-consolidated spunbonded layer (C).

The present invention further relates to a novel pre-consolidated spunbonded web defined in independent claim 25, to a novel composite nonwoven defined in independent claim 35, and to a novel hydroentangled absorbent composite nonwoven defined in independent claim 37.

The present invention further relates to a novel continuous system defined in independent claim 48, for producing the hydroentangled absorbent composite nonwoven of the invention.

The composite nonwoven of the invention can be used advantageously in all applications, where the absorption of liquid is needed.

The composite nonwoven of the invention is preferably used, but not only, in the field of hygienic industry, for making absorbent hygienic products. The present invention thus further relates to the use of this novel composite nonwoven for making hygienic products, and more particularly dry wipes, or wet wipes, or diapers, or training pants, or sanitary napkins, or incontinence products.

Additional and optional characteristics of the invention are also defined in the attached claims.

### BRIEF DESCRIPTION OF THE DRAWINGS

The characteristics and advantages of the invention will appear more clearly on reading the following detailed description which is made by way of non-exhaustive and non-limiting example, and with reference to the accompanying drawings, in which:

FIG. 1 is a general and schematic drawing of an example of absorbent composite nonwoven of the invention,

FIG. 2 is a schematic drawing of a first continuous system for producing the composite nonwoven of FIG. 1, and

FIG. 3 is a schematic drawing of a second continuous system for producing the composite nonwoven of FIG. 1,

FIG. 4 is a schematic drawing of a third continuous system for producing the composite nonwoven of FIG. 1,

FIG. 5 is plane view of an example of micro-bonding pattern that is suitable for practising the invention and that is referred as C#1 in the following detailed description,

FIG. 6 is a view in cross section of the micro-bonding pattern of FIG. 5, in plane VI-VI of FIG. 5,

FIG. 7 is a view in cross section of the micro-bonding pattern of FIG. 5, in plane VII-VII of FIG. 5,

FIG. 8 is plane view of an example of an other bonding pattern that is referred as C#2 in the following detailed description,

FIG. 9 is a view in cross section of the bonding pattern of FIG. 8, in plane IX-IX of FIG. 8,

FIG. 10 is a photography of a sample of pre-consolidated spunbonded web (A) of the invention taken with a microscope,

FIG. 11 is a photography of a sample of composite nonwoven of the invention (Spunbonded/Pulp/Carded) described hereafter and taken with a microscope on the spunbonded side (A) of the composite,

FIGS. 12A to 12H are different examples of spun filaments cross-sections that are suitable for practising the invention,

FIG. 13 is a photography of a sample of composite nonwoven of the invention (Spunbonded/Carded) described hereafter and taken with a microscope on the spunbonded side (A) of the composite.

### DETAILED DESCRIPTION

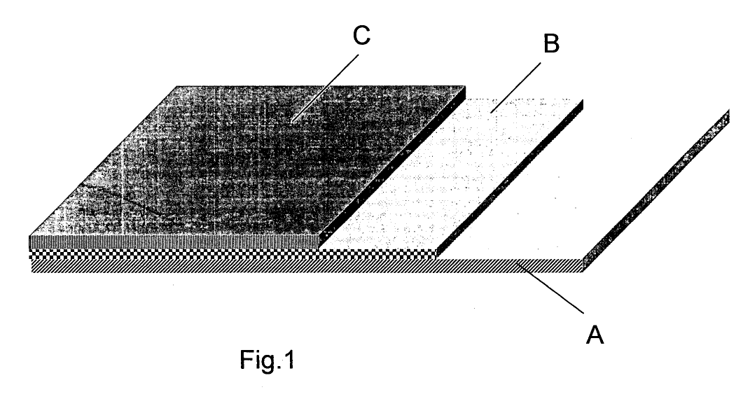

Referring to FIG. 1, a hydroentangled absorbent composite nonwoven of the invention comprises at least two superposed layers: a pre-consolidated spunbonded web A, and an absorbent pulp layer B, that is adjacent to said pre-consolidated spunbonded web A. In the preferred embodiment of FIG. 1, the composite nonwoven optionally comprises a nonwoven cover layer C adjacent to the absorbent pulp layer B, said pulp layer B being sandwiched between the spunbonded web A and the nonwoven cover layer C.

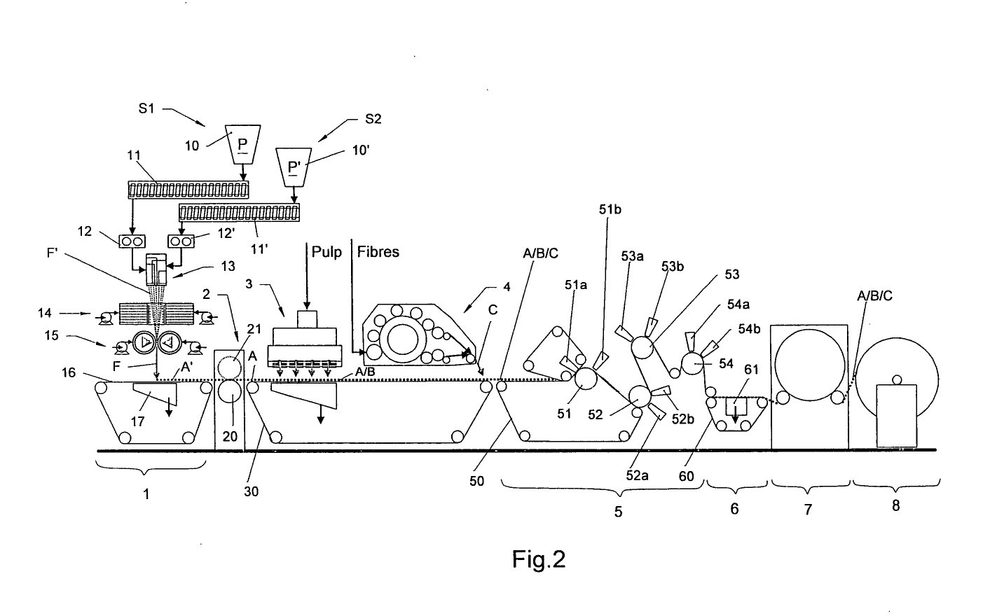

The composite nonwoven (A/B/C) of FIG. 1 is, for example, advantageously manufactured by means of the continuous system of FIG. 2.

The continuous system of FIG. 2 comprises a spunbonding unit 1, a thermal bonding unit 2, an air-laid unit 3, a carding unit 4, a hydraulic needling unit 5, a dewatering unit 6, a drying unit 7, and a winding unit 8.

### Spunbonding Unit 1

The spunbonding unit 1 is used for producing a non-consolidated spunbonded web A′ made of continuous spun filaments F.

The spunbonding unit 1 comprises at least one supplying line S1. Said supplying line S1 comprises a feeding hopper 10, an extruder 11 and metering pumps 12. The feeding hopper 10 contains a polymeric material P (for example in the form of pellets, or chips, or granulates, . . . ). Said hopper 10 is connected to the inlet of the extruder 11, that enables to continuously heat up and molten the polymeric material P. The outlet of the extruder 11 is connected to the inlet of metering pumps 12, via a distribution manifold. The outlets of the metering pumps 12 are connected to the inlet of a spinning pack 13. The metering pumps 12 are used for continuously dosing the molten polymer P into the spinning pack 13. This spinning pack 13 is used for producing a curtain of continuous filaments F′.

In the particular example of FIG. 1, the spunbonding unit 1 further comprises a second supplying line S2 for feeding the spinning pack 13 with a polymeric material P′. This second supplying line S2 comprises a feeding hopper 10′, that contains the polymeric material P′, an extruder 11′, and metering pumps 12′.

Downstream the spinning pack 13, the spunbonding unit 1 comprises an air quenching box 14 that is being used to cool down the filaments F′ issued from the spinning pack 13, and an air drawing equipment 15 that is being used to reduce the diameter of the filaments in order to form a curtain of filaments F having a smaller diameter.

The polymeric material(s) [P and/or P′] used for making the continuous spun filaments F can be any known spinnable polymeric material, and for example, polyolefin (in particular polypropylene or polyethylene), polyester, or polyamide, or any biodegradable thermoplastic polymer, like for example polylactic acid (PLA), or any blend thereof, or any copolymers thereof, or any blend of copolymers thereof.

These continuous spun filaments F can be, for example, monocomponent or multicomponent filaments, especially bicomponent filaments, and more especially sheath/core bicomponent filaments. When monocomponent spun filaments F are produced, only one supplying line (S1 or S2) can be used or both supplying lines can be used (S1 and S2). When bicomponent spun filaments F are produced, both supplying lines S1 and S2 are used simultaneously. In case of sheath/core bicomponent filaments, it is preferred, but not mandatory, to use polyethylene/polypropylene filaments.

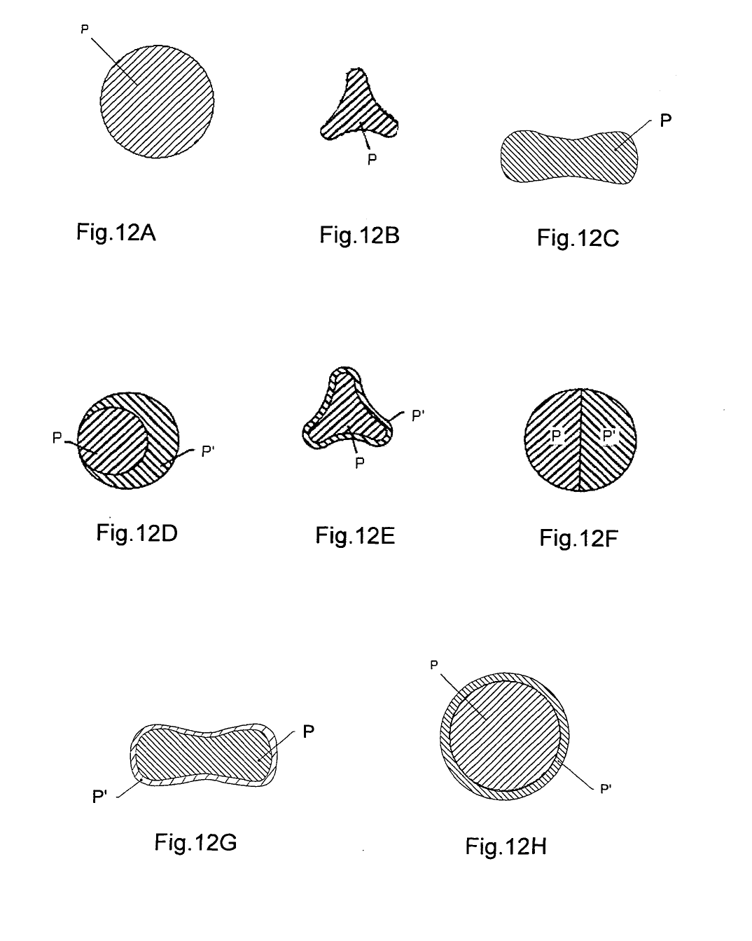

Various shapes in cross section for the filaments F can also be envisaged (round shape, oval shape, bilobal shape, trilobal shape, etc. . . . ). The shape in cross section of the continuous spun filament F is determined by the geometry of the holes of the spinneret plate of the spinning pack 13. Some non-limiting examples of different cross sections for monocomponent filaments that are suitable for the invention are illustrated on FIGS. 12A, 12B and 12C and some non-limiting examples of different cross sections for bi-component filaments that are suitable for the invention are illustrated on FIGS. 12D, 12E, 12F, 12G, 12H.

The air drawing equipment 15 is mounted above a movable and foraminous surface, such as a wire conveyor belt 16. The spun filaments F of reduced diameter, that are issued from the air drawing equipment 15, are laid down onto the said movable surface 16, where vacuum is applied, opposite to the filaments lay down side, by means of a vacuum box 17. A non-consolidated spunbonded web A′ made of continuous spun filaments F is thus formed on the surface of the belt 16, and is transported by the belt 16 towards the thermal bonding unit 2, that is mounted downstream the spunbonding unit 1.

According to a main characteristic of the invention, at least part of these continuous spun filaments F, preferably more than 50% of these continuous filaments F, and more preferably all these continuous filaments F, are microfilaments having a diameter DI less than or equal to 15 μm, and more preferably less than 10 μm.

Preferably, but not necessarily, the spunbonded web A′ is a light web whose weight is between 7 g/m² and 35 g/m², preferably less than 25 g/m², more preferably less than 15 g/m², and even more preferably less than 12 g/m².

The spunbonding unit 1 is knowingly set up by one skilled in the art, in order to produce such a light spunbonded web A′ made of continuous spun filaments F, that comprise or are constituted by very fine spun microfilaments having a diameter DI less than or equal to 15 μm, and more preferably less than or equal to 10 μm.

### Diameter of a Filament

Whatever the shape in cross section of a filament F is, the diameter DI of said filament F can be checked as follows. A sample of a filament F is being collected (for example on the belt 16), and the filament count CT is measured by applying the following known gravimetric method: the length of the sample is measured and the sample is being weighted. The weight of the sample, expressed in g (grams), is then correlated to the weight of 10000 meters of filament, in order to obtain the filament count (in dtex). The density d of the polymeric material being known, the diameter DI (in μm) of the continuous filament F is then calculated by using the following equation (1).

$$DI = \sqrt{\frac{400 \cdot CT}{π \cdot d}} \qquad ( 1 )$$

### Wherein

CT is the filament count in dtex:
d is the density (in g/cm³ or Kg/dm³) of the polymeric material

In case of a monocomponent filament, the density d of the polymer is well-known in the art. By way of examples only, the density of several homopolymer that are suitable for the invention are the following:

Polypropylene (PP): d=0.91 g/cm³
Polyethylene (PE): d=0.95 g/cm³
Polyethylene terephtalate (PET): d=1.37 g/cm³
Polylactic acid (PLA): d=1.25 g/cm³

In case for example of bicomponent filaments made of two different polymers P1 and P2, having known densities d1 and d2, the density d can be calculated with the following formula:

$$d = \frac{K 1}{K 1 + K 2} \times d 1 + \frac{K 2}{K 1 + K 2} \times d 2 \qquad ( 2 )$$

### Wherein:

K1 (is the mass flow of polymer P1 (expressed for example in Kg/h) measured by the dosing system installed between the hopper (10 or 10′) and the extruder (11 or 11′)
K2 (is the mass flow of polymer P2 (expressed for example in Kg/h) measured by the dosing system installed between the hopper (10 or 10′) and the extruder (11 or 11′)

In case of filaments having a round shape in cross section (monocomponent or multicomponent filaments), the diameter DI of the filament can also be measured by using an optical or electronic microscope. In that case, depending on the fiber diameter uniformity, it is recommended to perform several measurements of the filament diameter at different locations along the sample length, and to calculate an average value for the diameter DI.

### Thermal Bonding Unit 2

Referring to FIG. 2, the spunbonded web A′ is fed to a thermal bonding unit 2, that is being used in order to pre-consolidate the spunbonded web A′ by heat and mechanical compression (thermo-bonding), and form the pre-consolidated spunbonded web A of the composite nonwoven of FIG. 1.

In the particular example of FIG. 2, said thermal bonding unit 2 is a calender that comprises two heated pressure rolls 20, 21. The lower roll has a smooth surface, and is for example a smooth steel roll. The upper roll 21 has an engraved surface with protruding ribs, that are regularly distributed over the whole surface of the roll, and that form a micro-bonding pattern.

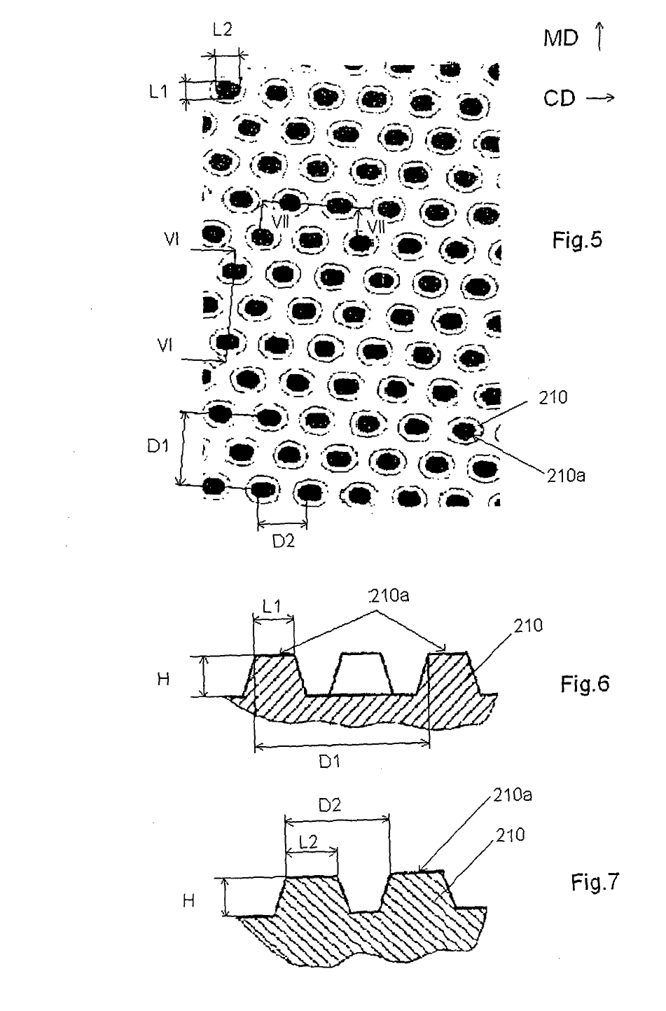

An example of a micro-bonding pattern for roll 21 that is suitable for practicing the invention is shown on FIGS. 5 to 7. This micro-bonding pattern will be referred hereafter “C#1”. The upper surface 210a of each protruding rib 210 forms one bonding dot.

The heating temperature of said rolls 20, 21 is set up in order to obtain a softening of the surface of the filaments F. The mechanical pressure exerted by the rolls on the spunbonded web is sufficient in order to obtain a thermo-bonding of the spun filaments F, under heat and pressure.

According to a main characteristic of the invention, the density of the bonding dots 210a (i.e. the number of bonding dots 210a per cm²) of the upper engraved roll 21 is very high, and at least equal to 90 bonding dots/cm², and more preferably at least equal to 100 bonding dots/cm²; the bonding ratio is low, and preferably less than 30% and more preferably less than 20%. The bonding ratio R is given by the following formula:

R=DD×DA×100 (3)

### Wherein:

DD is the density of bonding dots 210a (dots/cm²)
DA A is the area of one bonding dot 210 (cm²)

More particularly, the area DA of each bonding dot 210a is less than 0.5 mm², preferably less than 0.3 mm², and more preferably less than 0.2 mm².

In the particular example of FIGS. 5 to 7, the bonding dots 210a of the bonding pattern “C#1” have the same oval shape and the main dimensions of said bonding pattern are the followings:

Density of bonding dots (DD): 102 dots/cm²
Dot area (DA): 0.181 mm²
Bonding ratio (R): 18.5%
Dot length L1 (Machine Direction): 0.40 mm
Dot width L2 (Cross Direction): 0.54 mm
Dot height (H): 0.40 mm
Distance (D1) between two adjacent dots in Machine Direction (MD): 1.78 mm
Distance (D2) between two adjacent dots in Cross Direction (CD): 1.1 mm

The invention is however not limited to this particular bonding pattern of FIGS. 5 to 7. In particular, the bonding dots 210a can have different shapes (round shape, square shape, rectangular shape, etc. . . . ), and one bonding pattern can be constituted by a combination of bonding dots 210a of different shapes.

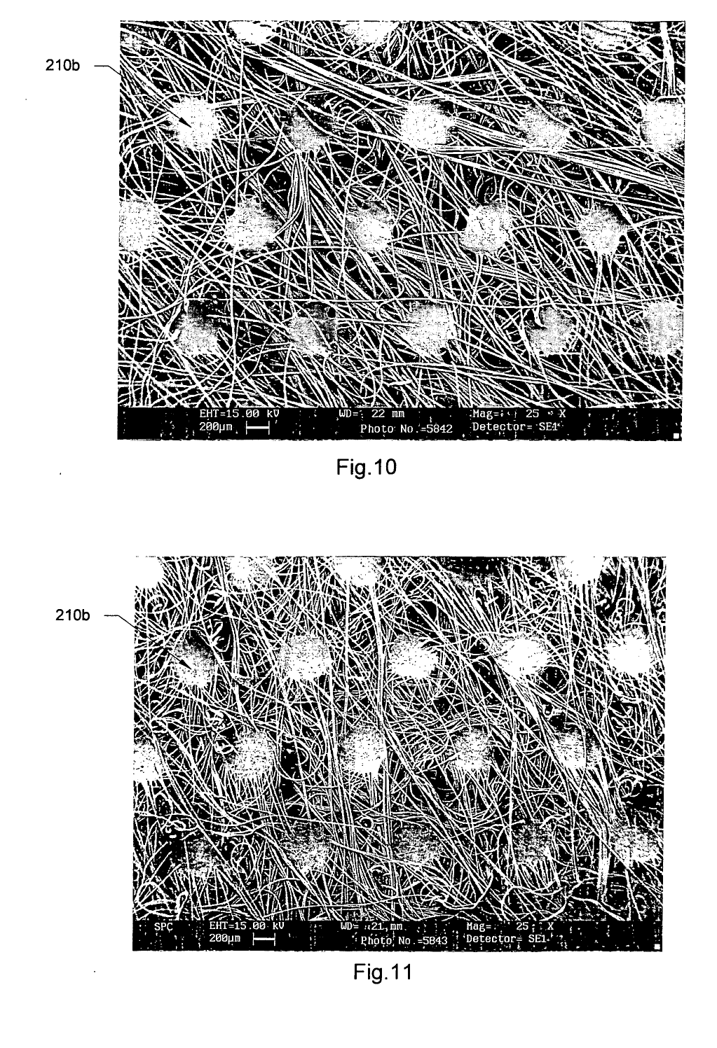

FIG. 10 shows a photography of an example of a pre-consolidated spunbonded web A of the invention, having a basis weight of 14 g/m². This pre-consolidated spunbonded web is made of spun filaments (F) that are made of homopolymer of polypropylene, and that have the so-called “papillon” cross section of FIG. 12C, and a diameter DI of 12 μm. This spunbonded web was thermo-bonded with a calendar unit 2, using the aforesaid microbonding pattern C#1.

Referring to this FIG. 10, the pre-consolidated spunbonded layer A, issued from the thermal bonding unit 2, comprises a high number of very small bonded dots 210b, that corresponds to the bonding pattern of the engraved roll 21, and wherein the spun microfilaments are locally fused at their surface. The density (DD) of these bonded dots 210b is the same than the density of the bonding dots 210a (≧90 bonded dots/cm²). The area of each bonded dots 210b of the pre-consolidated spunbonded layer A is preferably equal or less than the area of the corresponding bonding dot 210a of the bonding pattern. The bonding ratio (R′) of the pre-consolidated spunbonded layer A is preferably equal or less than the bonding ratio (R) of the bonding pattern. This bonding ratio R′ is given by the following formula:

$${R}^{′} = \frac{{\sum }^{ } {S}_{i}}{S} \times 100 \qquad ( 4 )$$

### Wherein:

S is whole area of a sample of the pre-consolidated spunbonded layer (A),
Sᵢ is the area of each individual bonded dot of said sample

### Air-Laid Unit 3

The traditional air-laid unit 3, which is mounted downstream the thermal bonding unit 2, is disclosed in details, for example, in European patent application EP 0 032 772. Said air-laid unit 3 is fed with loose pulp fibers, and more preferably with short wood pulp fibers.

The pre-consolidated spunbonded web A issued from thermal bonding unit 2 is transferred continuously to a second belt 30 where pulp fibers are laid down, using a conventional air-laid process, by means of said traditional air-laid unit 3.

Preferably, the air-laid unit 3 is set up in order to produce a pulp layer B whose weight is between 15 g/m² and 50 g/m².

At the output of the air-laid unit 3, a composite (A/B) made of a pre-consolidated spunbonded web A and of an absorbent pulp top layer B is obtained.

### Carding Unit 4

The carding unit 4, which is mounted between the air-laid unit 3 and the hydraulic needling unit 5, is used for producing in line a carded nonwoven cover layer C. Said carded nonwoven cover layer C issued from the carding unit 4 is laid down onto the top surface of the absorbent pulp layer B of the composite nonwoven (A/B) issued from the air-laid unit 3.

Preferably, the carding unit 4 is set up in order to produce a carded layer C, whose weight is between 10 g/m² and 30 g/m².

### Hydraulic Needling Unit 5

The composite nonwoven (A/B/C) is transported, downstream the carding unit 3, by means of a third conveyor belt 50 through the hydraulic needling unit 5. This hydraulic needling unit 5 is used for consolidating the nonwoven composite (A/B/C), by means of high pressure water jets (hydroentanglement process) that are directed at least towards the surface of the top layer (cover layer C), and that penetrate through the structure of the composite and are partially reflected back to the structure, in order to bind the layers (A, B and C) together.

In the particular example of FIG. 2, the water needling process is performed on both sides of the composite nonwoven (A/B/C).

More particularly, in the example of FIG. 2, the hydraulic needling unit 5 comprises four successive perforated drums. First perforated drum 51 is associated with two successive hydro-jet beams 51a and 51b. Second perforated drum 52 is associated with two successive hydro-jet beams 52a and 52b. Third perforated drum 53 is associated with two successive hydro-jet beams 53a and 53b. Fourth perforated drum 54 is associated with two successive hydro-jet beams 54a and 54b. The water pressure of the upstream hydro-jet beam 51a is lower than the water pressure of all the other downstream hydro-jet beams 51b, 52a, 52b, 53a, 53b, 54a, 54b, in order to obtain a pre-hydroentanglement of the layers.

At the exit of hydraulic needling unit 5, a hydroentangled and absorbent composite A/B/C is obtained.

### Dewatering Unit 6

This hydroentangled and absorbent composite A/B/C is transported downstream the hydraulic needling unit 5 by the conveyor belt 60 of a dewatering unit 6, and over a vacuum box 61, that enables to remove by suction from the composite A/B/C most of the water that has been absorbed during the water needling process (conventional dewatering process).

The hydroentanglement unit 5 and the dewatering unit 6 can be integrated in the same industrial equipment.

### Drying Unit 7

The dewatered hydroentangled absorbent composite nonwoven A/B/C issued from the dewatering unit 6 is continuously fed through the oven of the drying unit 7, wherein heat is applied to the composite (for example by means of hot air), in order to remove the remaining water still contained within the composite nonwoven.

### Winding Unit 8

Then the dried composite nonwoven A/B/C is wound in the form of a roll, by means of the winding unit 8.

Preferably, but not necessarily, the weight of said hydroentangled and absorbent composite A/B/C is between 27 g/m² and 115 g/m².

### EXAMPLES N^{o} 1 to N^{o} 26

The invention will now be illustrated by the following non-limiting examples.

### Examples N^{o} 1 to N^{o} 18

### Pre-Consolidated Spunbonded Web

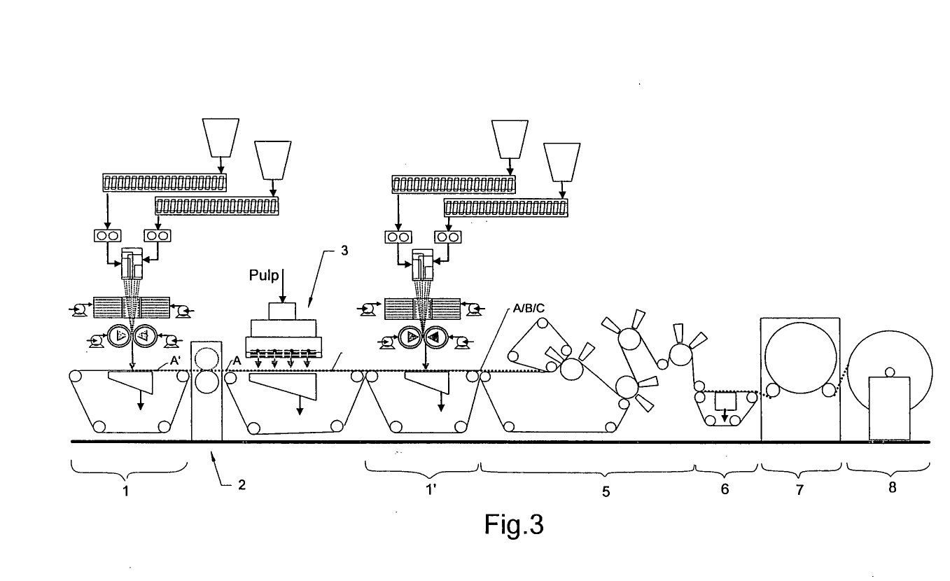

Several samples (Examples N^{o} 1 to N^{o} 18) of a pre-consolidated spunbonded web A were produced with the spunbonding unit 1 and thermal bonding unit 2 of FIG. 2 or 3. The main production data for each sample N^{o} 1 to 18 are summarized hereafter in tables 1A, 1B and 1C.

TABLE 1A
Spunbonded production data - Spinning

| | | | FILAMENT SPINNING [Filaments F′- Spunbonding unit (1)] | | | | |
|---|---|---|---|---|---|---|---|
| | | | Spinneret | | | | |
| | | | type (holes | | | | |
| | | | per meter/ | | | | Through |
| | RAW MATERIALS | | hole | filaments | filaments | filaments | put |
| | Polymer(s) | Polymer(s) | diameter) | section | diameter | count CT | (ghm) |
| Ex | type(s) | percentage(s) | [holes per | shape | DI | (dtex) | [g/hole/ |
| N^{o} | — | [%] | m/mm] | — | [μm] | [g/10000 m] | min] |
| 1 | PP⁽¹⁾ | 100 | 5000/0.35 | round | 9.5 | 0.65 | 0.33 |
| 2 | PP⁽¹⁾ | 100 | 5000/0.35 | round | 12.4 | 1.10 | 0.33 |
| 3 | PP⁽¹⁾ | 100 | 5000/0.35 | round | 13.5 | 1.30 | 0.33 |
| 4 | PP⁽¹⁾ | 100 | 5000/0.35 | round | 17.1 | 2.10 | 0.33 |
| 5 | PP⁽²⁾ | 100 | 6000/0.35 | round | 9.7 | 0.67 | 0.29 |
| 6 | PP⁽²⁾ | 100 | 6000/0.35 | round | 12.3 | 1.08 | 0.29 |
| 7 | PP⁽²⁾ | 100 | 6000/0.35 | round | 13.3 | 1.27 | 0.29 |
| 8 | PP⁽²⁾ | 100 | 6000/0.35 | round | 16.9 | 2.05 | 0.29 |
| 9 | PP⁽¹⁾ | 100 | 5000/0.35 | round | 9.7 | 0.67 | 0.35 |
| 10 | PP⁽¹⁾ | 100 | 5000/0.35 | round | 13.8 | 1.37 | 0.35 |
| 11 | PP⁽¹⁾ | 100 | 5000/0.35 | round | 16.7 | 2.00 | 0.35 |
| 12 | PP⁽²⁾ | 100 | 6000/0.35 | round | 9.6 | 0.66 | 0.29 |
| 13 | PP⁽²⁾ | 100 | 6000/0.35 | round | 13.6 | 1.33 | 0.29 |
| 14 | PP⁽²⁾ | 100 | 6000/0.35 | round | 17.1 | 2.10 | 0.29 |
| 15 | PP⁽²⁾ | 100 | 6000/0.35 | round | 13.6 | 1.33 | 0.297 |
| 16 | PP⁽²⁾ | 100 | 6000/0.35 | bilobal | 13.6 | 1.33 | 0.297 |
| 17 | PP⁽²⁾ | 100 | 6000/0.35 | round | 13.6 | 1.33 | 0.297 |
| 18 | PP⁽²⁾ | 100 | 6000/0.35 | bilobal | 13.6 | 1.33 | 0.297 |

PP⁽¹⁾: Polypropylene Borealis HH450FB
PP⁽²⁾: Polypropylene TOTAL PPH10099

TABLE 1B
Spunbonded production data - Web Forming

| | WEB FORMING [Filaments F- | | |
|---|---|---|---|
| | Spunbonding unit (1)] | | |
| | | | Nonwoven |
| | Draw slot | Line | Basis |
| Ex | Pressure | Speed | Weigth |
| N^{o} | [bar] | [m/min] | [g/m²] |
| 1 | 2.1 | 167 | 10 |
| 2 | 1.5 | 167 | 10 |
| 3 | 1.2 | 167 | 10 |
| 4 | 0.8 | 167 | 10 |
| 5 | 2 | 175 | 10 |
| 6 | 1.4 | 175 | 10 |
| 7 | 1.2 | 175 | 10 |
| 8 | 0.7 | 175 | 10 |
| 9 | 2.1 | 55 | 32 |
| 10 | 1.2 | 55 | 32 |
| 11 | 0.8 | 55 | 32 |
| 12 | 2 | 55 | 32 |
| 13 | 1.2 | 55 | 32 |
| 14 | 0.7 | 55 | 32 |
| 15 | 1 | 194 | 10 |
| 16 | 1 | 194 | 10 |
| 17 | 1 | 158 | 13 |
| 18 | 1 | 158 | 13 |

TABLE 1C
Spunbonded production data - Web Bonding

| | WEB BONDING [Thermal bonding unit (2)] | | | | | |
|---|---|---|---|---|---|---|
| | | | Calender | | | |
| | | Calender | Dots | Engraved | Smooth Roll | Calender |
| | Calender | Bonding | density | Roll (21) | (20) | Linear |
| | Type | Ratio (R) | (DD) | Temperature | Temperature | Pressure |
| Ex N^{o} | — | [%] | [dots/cm²] | [° C.] | [° C.] | [N/mm] |
| 1 | C#2 | 18 | 32.8 | 120 | 120 | 40 |
| 2 | C#2 | 18 | 32.8 | 120 | 120 | 40 |
| 3 | C#2 | 18 | 32.8 | 120 | 120 | 40 |
| 4 | C#2 | 18 | 32.8 | 120 | 120 | 40 |
| 5 | C#1 | 18.5 | 102 | 120 | 120 | 40 |
| 6 | C#1 | 18.5 | 102 | 120 | 120 | 40 |
| 7 | C#1 | 18.5 | 102 | 120 | 120 | 40 |
| 8 | C#1 | 18.5 | 102 | 120 | 120 | 40 |
| 9 | C#2 | 18 | 32.8 | 140 | 140 | 60 |
| 10 | C#2 | 18 | 32.8 | 140 | 140 | 60 |
| 11 | C#2 | 18 | 32.8 | 140 | 140 | 60 |
| 12 | C#1 | 18.5 | 102 | 140 | 140 | 60 |
| 13 | C#1 | 18.5 | 102 | 140 | 140 | 60 |
| 14 | C#1 | 18.5 | 102 | 140 | 140 | 60 |
| 15 | C#1 | 18.5 | 102 | 147 | 141 | 90 |
| 16 | C#1 | 18.5 | 102 | 147 | 141 | 90 |
| 17 | C#1 | 18.5 | 102 | 147 | 141 | 90 |
| 18 | C#1 | 18.5 | 102 | 147 | 141 | 90 |

In all examples N^{o} 1 to N^{o} 15, and N^{o} 17 the filaments of the spunbonded web were round monocomponent filaments made of homopolymer of polypropylene. In examples N^{o} 16 and 18, the filaments of the spunbonded web were bilobal monocomponent filaments made of homopolymer of polypropylene. The bilobal shape of the filaments (also called “papillon”) is well-known and shown on FIG. 12C. In Examples N^{o} 1 to N^{o} 8, and N^{o} 15 and 16 the weight of the spunbonded web was around 10 g/m²; In Examples N^{o} 9 to N^{o} 14, the weight of the spunbonded web was around 32 g/m². In Examples N^{o} 17 and 18, the weight of the spunbonded web was around 13 g/m².

### Examples N^{o} 5 to N^{o} 7 and Examples N^{o} 12 and N^{o} 13

### Invention

Examples N^{o} 5 to N^{o} 7 (10 g/m²) and examples N^{o} 12 and N^{o} 13 (32 g/m²) relate to pre-consolidated spunbonded webs of the invention, which have been compressed and thermo-bonded with the same engraved roll 21 having the previously described micro-bonding pattern “C#1”. They differ from each other by the diameter (DI) of their continuous spun microfilaments (F).

### Comparative Examples N^{o} 1 to 4 and N^{o} 8 to N^{o} 11, and N^{o} 14

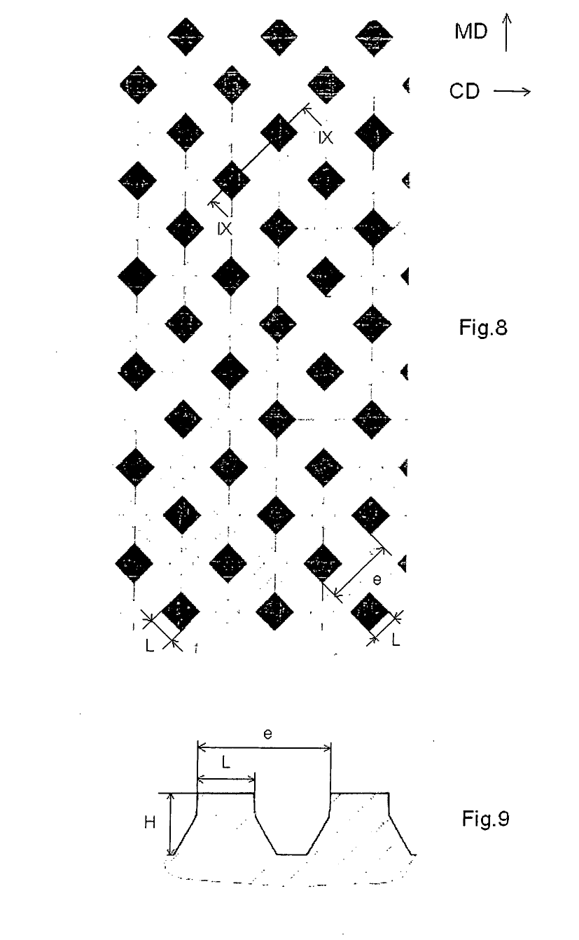

Examples N^{o} 1 to N^{o} 4 (10 g/m²) and examples N^{o} 9 to N^{o} 11 (32 g/m²) relate to spunbonded webs which have been compressed and thermo-bonded with the same engraved roll 21, said engraved roll 21 having the bonding pattern of FIGS. 8 and 9. The main dimensions of this bonding pattern (referred therein “C#2”) were the followings:

Shape of the bonding dots: square
Density of bonding dots: 32.8 dots/cm²
Bonding ratio (R): 18%
Dot width (L): 0.74 mm
Dot area (DA): 0.5476 mm²
Dot height (H): 0.8 mm
Distance (e) between two adjacent dots: 1.747 mm

In respect of the low density of bonding dots (32.8 dots/cm²) and of the large area of each bonding dots (0.5476 mm²), said bonding pattern “C#2” is outside the scope of the invention, and these examples N^{o} 1 to N^{o} 4 (10 g/m²) and examples N^{o} 9 to N^{o} 11 (32 g/m²) are thus comparative examples, not covered by the invention.

Example N^{o} 8 (10 g/m²) and example N^{o} 14 (32 g/m²) relate to pre-consolidated spunbonded webs, which have been thermo-bonded with microbonding pattern “C#1”, but which are made of continuous spun filaments (F), having a diameter (DI) higher than 15 μm (i.e. outside the scope of the invention).

### Air Permeability Test

For each example N^{o} 1 to N^{o} 14, the air permeability of the pre-consolidated spunbonded web was measured according to the following method.

Air Permeability Test was performed on a Textest model FX 3300 available from the Textest Instruments—Zurich, according to WSP 70.1 (05) standard. The rate of air flow passing perpendicularly through a given area of fabric is measured at a given pressure difference across the fabric test area over a given time period. The specimen (a single layer) was positioned in the circular specimen holder, with an orifice allowing the test to be carried out on an area of 38 cm². The pressure gap was set at 125 Pa.

The air permeability results (expressed in m³/m²/min) for examples N^{o} 1 to N^{o} 14 are summarized hereafter in tables 2A, 2B, 3A and 3B.

TABLE 2A
Spunbonded web - 10 g/m² - Air permeability versus spun filament
diameter

| | | | | NONWOVEN | | Air |
|---|---|---|---|---|---|---|
| | | | | | Equivalent Area | Permeability |
| | | | Filaments Lateral | | (Filaments | Ref. Method: |
| | | | Area (1 filament, | | Lateral Area in | WSP 70.1 |
| | Count | Diameter | length = 10000 m) | Basis | 1 m² of | (05) |
| | CT (dtex) | DI | C = (B/10⁶) × | weight | Nonwoven) | — |
| Ex | A | B | 10000 | D | E = (C/A) × D | [m³/m² × |
| N^{o} | [g/10000 m] | [μm] | [m²] | g/m² | [m²/m²] | min)] |
| 1 | 0.65 | 9.5 | 0.10 | 10 | 1.47 | 253 |
| 2 | 1.10 | 12.4 | 0.12 | 10 | 1.13 | 266 |
| 3 | 1.30 | 13.5 | 0.13 | 10 | 1.04 | 279 |
| 4 | 2.10 | 17.1 | 0.17 | 10 | 0.82 | 370 |

TABLE 2B
Spunbonded web - 32 g/m² - Air permeability versus spun filament diameter

| | | | | NONWOVEN | | |
|---|---|---|---|---|---|---|
| | FIBER | | | | Equivalent | |
| | | | Filaments | | Area | Air |
| | | | Lateral Area (1 | | (Filaments | Permeability |
| | | | filament, | | Lateral Area in | Ref. Method: |
| | | | length = 10000 m) | | 1 m² of | WSP 70.1 (05) |
| | count CT(dtex) | Diameter | C = (B/10⁶) × | Basis weight | Nonwoven) | — |
| Ex | A | B | 10000 | D | E = (C/A) × D | [m³/m² × |
| N^{o} | [g/10000 m] | [μm] | [m²] | g/m² | [m²/m²] | min)] |
| 9 | 0.67 | 947 | 0.10 | 32 | 4.63 | 77 |
| 10 | 1.37 | 13.8 | 0.14 | 32 | 3.23 | 88 |
| 11 | 2.00 | 16.7 | 0.17 | 32 | 2.68 | 101 |

TABLE 3A
Spunbonded web - 10 g/m² - Air permeability versus calender
design (population dots)

| | | | | | | Air |
|---|---|---|---|---|---|---|
| | | | | | | Permeability |
| | | | CALENDER (DESIGN) | | | Ref. Method: |
| | FILAMENT | NONWOVEN | | Bonding Ratio | Dots density | WSP 70.1 (05) |
| | Diameter (DI) | Basis weight | Type | (R) | (DD) | [m³/(m² × |
| Ex | [μm] | [g/m²] | — | [%] | [dots/cm²] | min)] |
| 1 | 9.5 | 10 | C#2 | 18.0 | 32.8 | 253 |
| 5 | 9.7 | 10 | C#1 | 18.5 | 102 | 224 |
| 2 | 12.4 | 10 | C#2 | 18.0 | 32.8 | 266 |
| 6 | 12.3 | 10 | C#1 | 18.5 | 102 | 240 |
| 3 | 13.5 | 10 | C#2 | 18.0 | 32.8 | 279 |
| 7 | 13.3 | 10 | C#1 | 18.5 | 102 | 261 |
| 4 | 17.1 | 10 | C#2 | 18.0 | 32.8 | 370 |
| 8 | 16.9 | 10 | C#1 | 18.5 | 102 | 329 |

TABLE 3B
Spunbonded web - 32g/m² - Air permeability versus calender
design (population dots)

| | | | | | | Air |
|---|---|---|---|---|---|---|
| | | | | | | Permeability |
| | | | CALENDER (DESIGN) | | | Ref. Method: |
| | FIBRE | NONWOVEN | | Bonding Ratio | Dots density | WSP 70.1 (05) |
| EX | diameter (DI) | Basis weight | Type | (R) | (DD) | [m³/(m² × |
| N^{o} | [μm] | [g/m²] | — | [%] | [dots/cm²] | min)] |
| 9 | 9.7 | 32 | C#2 | 18 | 32.8 | 77 |
| 12 | 9.6 | 32 | C#1 | 18.5 | 102 | 68 |
| 10 | 13.8 | 32 | C#2 | 18 | 32.8 | 88 |
| 13 | 13.6 | 32 | C#1 | 18.5 | 102 | 80 |
| 11 | 16.7 | 32 | C#2 | 18 | 32.8 | 101 |
| 14 | 17.1 | 32 | C#1 | 18.5 | 102 | 94 |

The air permeability measurements show that the air permeability of the pre-consolidated spunbonded web A increases with the spun filament diameter (DI) and decreases with the density (DD) of the bonding dots. Pre-consolidated spunbonded webs of the invention (Examples N^{o} 5, N^{o} 6, N^{o} 7, N^{o} 12 and N^{o} 13) advantageously exhibit lower air permeability, and the spunbonded layer constitutes therefore an improved barrier for the pulp fibres during the water needling process of the composite nonwoven.

### Examples N^{o} 15 to N^{o} 18

Different tests have been performed on pre-consolidated spunbonded webs produced accordingly to examples N^{o} 15 to 18. The results of these tests are given in Table 3C. The air permeability tests were performed accordingly to standard method WSP 70.1 (05) as previously described. The basis weight was measured accordingly to standard method WSP 130.1. The tensile property tests (CD_Tensile@peak; MD-Tensile@peak; CD_Elongation@peak; MD_Elongation@peak) were performed accordingly to standard method WSP 110.4 (05) as described hereafter for the composite nonwoven A/B/C. The opacity was measured accordingly to standard method WSP 60.4(05). The thickness (calliper) was measured accordingly to standard method WSP 120.6 (05).

TABLE 3C
round/bilobal

| | | | N^{o} 15 | N^{o} 16 | N^{o} 17 | N^{o} 18 |
|---|---|---|---|---|---|---|
| | | | (10gsm- | (10gsm- | (13gsm- | (13gsm- |
| | Test | | round- | bilobal- | round- | bilobal- |
| Parameter | Method | Units | C#1) | C#1) | C#1) | C#1) |
| Air permeability | WSP | m³/m²/min | 229 | 203 | 167 | 139 |
| | 70.1 | | | | | |
| | (05) | | | | | |
| Basis weight | WSP | g/m² | 10.6 | 9.8 | 13.7 | 13.4 |
| | 130.1 | | | | | |
| CD_Tensile@peak | WSP | N/inch | 4.08 | 5.17 | 9.34 | 9.59 |
| | 110.4 | | | | | |
| | (05) | | | | | |
| MD-Tensile@peak | WSP | N/inch | 13.31 | 13.98 | 23.34 | 24.21 |
| | 110.4 | | | | | |
| | (05) | | | | | |
| CD_Elongation@peak | WSP | % | 48.3 | 53.8 | 63.9 | 66.3 |
| | 110.4 | | | | | |
| | (05) | | | | | |
| MD_Elongation@peak | WSP | % | 38.3 | 43.6 | 59.8 | 61.7 |
| | 110.4 | | | | | |
| | (05) | | | | | |
| Opacity | WSP | % | 16.5 | 19.2 | 18.1 | 25.2 |
| | 60.4 | | | | | |
| | (05) | | | | | |
| Caliper | WSP | mm | 0.14 | 0.11 | 0.15 | 0.12 |
| | 120.6 | | | | | |
| | (05) | | | | | |

The results in Table 3C show that the low weight spunbonded webs that have been microbonded (pattern C#1) and that are made of bilobal filaments (N^{o} 16 and N^{o} 18) exhibit surprisingly a lower air permeability and higher opacity than low weight spunbonded webs that have been also microbonded (pattern C#1) but that are made of round filaments (N^{o} 15 and N^{o} 17). Even more surprisingly, the elongation at peak properties (both in CD and MD directions) are significantly improved when bilobal filaments are used.

### Examples N^{o} 19 to N^{o} 26

### Composite Nonwoven A/B/C

Several samples (Examples N^{o} 19 to N^{o} 26) of a three-layered hydroentangled and absorbent composite nonwoven (A/B/C) were produced by means of a continuous system like the one previously described and shown on FIG. 2. The main production data for these examples N^{o} 19 to 26 are summarized hereafter in tables 4A, 4B and 4C.

TABLE 4A
Composite nonwoven A/B/C (Spun/Pulp/Carded)-Production data

| | | | | | RAW MATERIALS | | |
|---|---|---|---|---|---|---|---|
| | | | | | | PULP (B) | |
| | SPC Basis Weight (A/B/C) | | | | SPUNBONDED (A) | Pulp | CARDED (C) |
| Ex | Total | A | B | C | Spunbonded type | type | Fibre(s) type(s) |
| N^{o} | [g/m²] | [g/m²] | [g/m²] | [g/m²] | Ex | — | — |
| 19 | 45 | 10 | 22 | 13 | N^{o} 2 | (I) | (II) |
| 20 | 45 | 10 | 22 | 13 | N^{o} 1 | (I) | (II) |
| 21 | 45 | 10 | 22 | 13 | N^{o} 5 | (I) | (II) |
| 22 | 45 | 10 | 22 | 13 | N^{o} 6 | (I) | (II) |
| 23 | 90 | 32 | 33 | 25 | N^{o} 10 | (I) | (III) |
| 24 | 90 | 32 | 33 | 25 | N^{o} 9 | (I) | (III) |
| 25 | 90 | 32 | 33 | 25 | N^{o} 13 | (I) | (III) |
| 26 | 90 | 32 | 33 | 25 | N 12 | (I) | (III) |

(I) Weyerhauser, NF 405
(II) PP Arborea Perm. Phil., 1.7 dtex, 38 mm
(III) PP Arborea Perm. Phil., 1.7 dtex, 38 mm (50% wt)/Lyocell, Lenzing Tencel, 1.7dtex, 38 mm (50% wt)

The basis weight of the composite nonwoven of examples N^{o} 19 to 22 was 45 g/m². The basis weight of the composite nonwoven of examples N^{o} 23 to 26 was 90 g/m².

Examples N^{o} 19, N^{o} 20, N^{o} 23 and N^{o} 24 relate to hydroentangled and absorbent composite nonwoven comprising a spunbonded layer A that is the same respectively than examples N^{o} 2, N^{o} 1, N^{o} 10 and N^{o} 9, and are thus comparative examples not covered by the invention.

Examples N^{o} 21, N^{o} 22, N^{o} 25 and N^{o} 26 relate to hydroentangled and absorbent composite nonwoven comprising a spunbonded layer A that is the same respectively than examples N^{o} 5, N^{o} 6, N^{o} 13 and N^{o} 12, and are thus covered by the invention.

TABLE 4B
Composite nonwoven A/B/C (Spun/Pulp/Carded)-
Hydroentanglement step

| | HYDROENTAGLEMENT (UNIT 5) | | | | | | | | |
|---|---|---|---|---|---|---|---|---|---|
| | Beam | Beam | Beam | Beam | Beam | Beam | Beam | Beam | Pattern |
| Ex. | (51a) | (51b) | (52a) | (52b) | (53a) | (53b) | (54a) | (54b) | type (54) |
| N^{o} | [bar] | [bar] | [bar] | [bar] | [bar] | [bar] | [bar] | [bar] | — |
| 19 | 15 | 30 | 70 | 70 | 45 | 45 | — | — | None |
| 20 | 15 | 30 | 65 | 65 | 50 | 50 | — | — | None |
| 21 | 15 | 35 | 90 | 90 | 70 | — | 70 | — | None |
| 22 | 15 | 35 | 90 | 90 | 70 | — | 70 | — | None |
| 23 | 15 | 50 | 130 | 130 | 70 | 70 | — | — | none |
| 24 | 15 | 50 | 130 | 130 | 70 | 70 | — | — | none |
| 25 | 20 | 80 | 70 | 70 | 100 | 100 | — | — | none |
| 26 | 20 | 80 | 70 | 70 | 100 | 100 | — | — | none |

TABLE 4C
Composite nonwoven A/B/C (Spun/Pulp/Carded)- Drying and
winding step

| | DRYING | WINDING |
|---|---|---|
| | (Unit 7) | (Unit 8) |
| | Oven | line |
| Ex. | Temperature | speed |
| N^{o} | [° C.] | [m/min] |
| 19 | 110 | 40 |
| 20 | 110 | 40 |
| 21 | 165 | 187 |
| 22 | 165 | 187 |
| 23 | 110 | 30 |
| 24 | 110 | 30 |
| 25 | 130 | 59 |
| 26 | 130 | 59 |

Different tests have been performed on samples of examples N^{o} 19 to 26.

### Abrasion Resistance Test

Abrasion resistance testing was performed on a Martindale Abrasion Tester (Model: Nu-Martindale Abrasion and Pilling tester from James H. Heal & Co. Ltd—Halifax, England). Tests were performed according to ASTM D 4966-98 using a pressure of 12 kilopascals (KPa) on the spunbonded side (A) of the composite A/B/C.

Samples were subjected to 150 cycles and were then examined for the presence of surface fuzzing, pilling, roping or holes. The samples were compared to a visual scale and assigned a wear number from 1 to 5, wherein wear number 5 was indicating little or no visible abrasion and wear number 1 was indicating a hole worn through the sample.

The results of the abrasion resistance test are summarized hereafter in table 5A (basis weight 45 g/m²) and in table 5B (basis weight 90 g/m²).

TABLE 5A
Composite SPC - 45 g/m² - Abrasion resistance

| | | | | | MARTINDALE |
|---|---|---|---|---|---|
| | | | | | ABRASION |
| | | | | | Ref. Method: |
| | | | | | ASTM D4966-98 |
| | | SPUNBONDED (A) | | | Fabric side |
| | | | | Calender | tested: |
| SPC (A/B/C) | | | Filament | Dots density | spunbonded |
| Ex. | Basis weight | A | diameter | (DD) | [values: from |
| N^{o} | [g/m²] | Ex. | (DI) [μm] | [dots/cm²] | 1 to 5] |
| 19 | 45 | N^{o} 2 | 12.4 | 32.8 | 3.25 |
| 20 | 45 | N^{o} 1 | 9.5 | 32.8 | 3.50 |
| 21 | 45 | N^{o} 6 | 12.3 | 102 | 4.00 |
| 22 | 45 | N^{o} 5 | 9.7 | 102 | 4.25 |

TABLE 5B
Composite SPC - 90 g/m² - Abrasion resistance

| | | | | | MARTINDALE |
|---|---|---|---|---|---|
| | | | | | ABRASION |
| | | | | | Ref. Method: |
| | | | | | ASTM D4966-98 |
| | | SPUNBONDED | | | Fabric side |
| | | | | Calender | tested: |
| SPC (A/B/C) | | | Filament | Dots density | spunbonded |
| Ex. | Basis weight | A | diameter | (DD) | [values: from |
| N^{o} | [g/m²] | Ex. | (DI) [μm] | [dots/cm²] | 1 to 5] |
| 23 | 90 | N^{o} 10 | 13.8 | 32.8 | 4.00 |
| 24 | 90 | N^{o} 9 | 9.7 | 32.8 | 4.25 |
| 25 | 90 | N^{o} 13 | 13.6 | 102 | 4.50 |
| 26 | 90 | N^{o} 12 | 9.6 | 102 | 4.75 |

The pre-consolidated spunbonded layers A of examples N^{o} 21, 22, and 26 (invention) have advantageously a better abrasion resistance than the pre-consolidated spunbonded layers of the other comparative examples. The comparison between examples having the same basis weight and having spun filaments of similar diameter (Ex. N^{o} 19 vs. Ex. N^{o} 23; EX. N^{o} 20 vs. Ex. N^{o} 21; EX. N^{o} 23 vs. Ex. N^{o} 25; EX. N^{o} 24 vs. Ex. N^{o} 26) further shows that the microbonding pattern of the invention, with very high density of bonding dots (DD), improves the abrasion resistance of the spunbonded layer A, compared with the use of a bonding pattern having a low density of bonding dots (DD).

### Handle-O-Meter Test

Handle-O-Meter testing was performed on a Handle-O-Meter Model no. 211-5, model 211-2001 available from the Thwing-Albert Company. Tests were performed according to WSP 90.3.0 (05) standard. The nonwoven to be tested is deformed through a restricted opening by a plunger and the required force corresponds to the surface friction of the nonwoven.

The determination of the combined effects of stiffness and thickness is correlated with finished product properties like softness. A slot width of 6.4 mm and square specimen (200 mm×200 mm) were used. Three specimens for each sample were tested. Each specimen was tested on both sides, spunbonded (side1) and carded (side2) and in both directions, Machine directions (MD) and Cross direction (CD). Therefore, the total number of measurements for each specimen was 4. Results include average value MD and CD (side1, side 2) and the “Total Hand” (sum of: MD side1, MD side2, CD side1, CD side2).

The results of the handle-o-meter test are summarized hereafter in table 6A (basis weight 45 g/m²) and in table 6B (basis weight 90 g/m²). The composite nonwovens (A/B/C) of examples N^{o} 21, 22, 25 and 26 (invention) have advantageously a lower stiffness both in CD and MD directions (and thus a higher softness) than the other comparative examples. The comparison between examples having the same basis weight and having spun filaments of similar diameter (Ex. N^{o} 19 vs. Ex. N^{o} 22; EX. N^{o} 20 vs. Ex. N^{o} 21; EX. N^{o} 23 vs. Ex. N^{o} 25; EX. N^{o} 24 vs. Ex. N^{o} 26) shows that the microbonding pattern of the invention, with very high density of bonding dots (DD), improves the softness of the final product, compared with the use of a bonding pattern having a low density of bonding dots (DD).

TABLE 6A
Composite SPC - 45 g/m² - stiffness (handle-o-meter test)

| | HANDLE-O-METER Ref. Method: WSP 90.3.0 (05) | | | | | | | |
|---|---|---|---|---|---|---|---|---|
| SPC | | Side 1 up, | Side 1 up, | Side 2 up, | Side 2 up, | Average, | Average, | |
| (A/B/C) | | MD | CD | MD | CD | MD | CD | Total “Hand” |
| Ex | Specimen | A1 | B1 | C1 | D1 | (A1 + C1)/2 | (B1 + D1)/2 | A1 + B1 + C1 + D1 |
| N^{o} | N | [cN] | [cN] | [cN] | [cN] | [cN] | [cN] | [cN] |
| 19 | 1 | 60.8 | 15.2 | 70.2 | 12.6 | 65.5 | 13.9 | 158.8 |
| | 2 | 67.1 | 11.8 | 62.7 | 11.9 | 64.9 | 11.9 | 153.5 |
| | 3 | 74.4 | 12.8 | 69.1 | 12.1 | 71.8 | 12.5 | 168.4 |
| | average | | | | | 67.4 | 12.7 | 160.2 |
| 20 | 1 | 51.9 | 12.1 | 54.1 | 10.1 | 53.0 | 11.1 | 128.2 |
| | 2 | 58.0 | 11.5 | 52.0 | 8.6 | 55.0 | 10.1 | 130.1 |
| | 3 | 57.8 | 11.9 | 52.1 | 9.0 | 55.0 | 10.5 | 130.8 |
| | average | | | | | 54.3 | 10.5 | 129.7 |
| 21 | 1 | 49.0 | 10.3 | 49.3 | 7.7 | 49.2 | 9.0 | 116.3 |
| | 2 | 52.1 | 10.7 | 51.1 | 7.3 | 51.6 | 9.0 | 121.2 |
| | 3 | 50.0 | 11.0 | 50.4 | 9.0 | 50.2 | 10.0 | 120.4 |
| | average | | | | | 50.3 | 9.3 | 119.3 |
| 22 | 1 | 44.5 | 9.9 | 49.2 | 6.8 | 46.9 | 8.4 | 110.4 |
| | 2 | 43.0 | 9.0 | 48.0 | 6.7 | 45.5 | 7.9 | 106.7 |
| | 3 | 47.1 | 9.3 | 46.2 | 7.0 | 46.7 | 8.2 | 109.6 |
| | average | | | | | 46.3 | 8.1 | 108.9 |

TABLE 6B
Composite SPC - 90 g/m² - stiffness (handle-o-meter test)

| | HANDLE-O-METER Ref. Method: WSP 90.3.0 (05) | | | | | | | |
|---|---|---|---|---|---|---|---|---|
| SPC | | Side 1 | Side 1 up, | Side 2 | Side 2 | Average, | Average, | Total “Hand” |
| (A/B/C) | | up, MD | CD | up, MD | up, CD | MD | CD | A1 + B1 + C1 + |
| EX | Specimen | A1 | B1 | C1 | D1 | (A1 + C1)/2 | (B1 + D1)/2 | D1 |
| N^{o} | N | [cN] | [cN] | [cN] | [cN] | [cN] | [cN] | [cN] |
| 23 | 1 | 91.9 | 19.8 | 94.0 | 21.1 | 93.0 | 20.5 | 226.8 |
| | 2 | 90.1 | 18.4 | 93.1 | 23.3 | 91.6 | 20.9 | 224.9 |
| | 3 | 90.0 | 18.7 | 93.3 | 22.0 | 91.7 | 20.4 | 224.0 |
| | average | | | | | 92.1 | 20.6 | 225.2 |
| 24 | 1 | 74.0 | 16.1 | 80.0 | 17.0 | 91.8 | 20.6 | 187.1 |
| | 2 | 70.3 | 15.8 | 81.1 | 16.4 | 75.7 | 16.1 | 183.6 |
| | 3 | 72.4 | 15.9 | 78.5 | 17.9 | 75.5 | 16.9 | 184.7 |
| | average | | | | | 81.0 | 17.9 | 185.1 |
| 25 | 1 | 31.7 | 12.8 | 53.3 | 15.7 | 42.5 | 14.3 | 113.5 |
| | 2 | 32.9 | 13.1 | 49.3 | 17.1 | 41.1 | 15.1 | 112.4 |
| | 3 | 32.9 | 13.1 | 49.3 | 17.1 | 41.1 | 15.1 | 112.4 |
| | average | | | | | 41.6 | 14.8 | 112.8 |
| 26 | 1 | 29.0 | 11.4 | 47.3 | 14.0 | 41.3 | 15.0 | 101.7 |
| | 2 | 28.4 | 12.0 | 49.3 | 14.5 | 41.3 | 15.0 | 104.2 |
| | 3 | 27.3 | 13.2 | 47.1 | 13.4 | 41.4 | 14.9 | 101.0 |
| | average | | | | | 41.3 | 15.0 | 102.3 |

### Tensile Properties Test

Tensile tests were performed on a dynamometer model 5564 available from Instron Instruments, according to WSP 110.4 (05) standard.

Tensile strength refers to the maximum load (i.e., peak load) encountered while elongating the sample to break. Elongation at peak is the specimen elongation that corresponds to the peak load. Measurements were made in cross-direction on dry samples.

Specimens were cut 25 mm width and 125 mm length. The distance between the clamps of the dynamometer was set at 75 mm and the traction speed was set at 300 mm/min.

The results of the tensile properties test are summarized hereafter in table 7A (basis weight 45 g/m²) and in table 7B (basis weight 90 g/m²).

TABLE 7A
Composite SPC - 45 g/m² - Tensile properties (MD, CD)

| | | SPUNBONDED | | | TENSILE PROPERTIES Ref. Method: WSP | | | |
|---|---|---|---|---|---|---|---|---|
| | | | | Calender | 110.4 (05) | | | |
| SPC (A/B/C) | | | Filament | Dots | | | Elongation | Elongation |
| | Basis | | diameter | density | Load at | Load at | at Peak, | at Peak, |
| Ex | weight | A | (DI) | (DD) | Peak, MD | Peak, CD | MD | CD |
| N^{o} | [g/m²] | EX | [μm] | [dots/cm²] | [N] | [N] | [%] | [%] |
| 19 | 45 | N^{o} 2 | 12.4 | 32.8 | 49.0 | 5.6 | 57 | 73 |
| 20 | 45 | N^{o} 1 | 9.5 | 32.8 | 53.3 | 8.9 | 60 | 123 |
| 21 | 45 | N^{o} 6 | 12.3 | 102 | 50.1 | 9.0 | 62 | 110 |
| 22 | 45 | N^{o} 5 | 9.7 | 102 | 57.0 | 9.5 | 59 | 127 |

TABLE 7B
Composite SPC - 45 g/m² - Tensile properties (MD, CD)

| | | SPUNBONDED | | | TENSILE PROPERTIES Ref. Method: WSP | | | |
|---|---|---|---|---|---|---|---|---|
| | | | | Calender | 110.4 (05) | | | |
| SPC (A/B/C) | | | Filament | Dots | | | Elongation | Elongation |
| | Basis | | diameter | density | Load at | Load at | at Peak, | at Peak, |
| EX | weight | A | (DI) | (DD) | Peak, MD | Peak, CD | MD | CD |
| N^{o} | [g/m²] | EX | [μm] | [dots/cm²] | [N] | [N] | [%] | [%] |
| 23 | 90 | N^{o} 10 | 13.8 | 32.8 | 82.7 | 19.3 | 35 | 54 |
| 24 | 90 | N^{o} 9 | 9.7 | 32.8 | 87.1 | 31.1 | 62 | 99 |
| 25 | 90 | N^{o} 13 | 13.6 | 102 | 92.2 | 39.1 | 79 | 104 |
| 26 | 90 | N^{o} 12 | 9.6 | 102 | 96.3 | 43.1 | 82 | 111 |

The comparison between examples having the same basis weight and having spun filaments of similar diameter (Ex. N^{o} 19 vs. Ex. N^{o} 22; EX. N^{o} 20 vs. Ex. N^{o} 21; EX. N^{o} 23 vs. Ex. N^{o} 25; EX. N^{o} 24 vs. Ex. N^{o} 26) shows that the microbonding pattern of the invention, with very high density of bonding dots (DD), improves these tensile properties of the final product, especially load at peak both in CD and MD directions, compared with the use of a bonding pattern having a low density of bonding dots (DD).

In addition to the above properties, it has been further noticed that during the water needling process of the composite nonwovens (A/B/C) of examples N^{o} 21, 22, 25 and 26 (invention), the pulp fibres forming the absorbent layers were only slightly washed through the pre-consolidated spunbonded layer A, and were thus retained for the useful effect of the product. This reduction of pulp loss can be explained by the very small pore size (or otherwise stated the lower air permeability) of the said pre-consolidated spunbonded layer (A) of the invention.

By way only of non-limiting example, FIG. 11 shows a photography of a sample of a hydroentangled absorbent composite nonwoven (A/B/C) of the invention, having a basis weight of 50 g/m², and wherein.
layer A is a pre-consolidated spunbonded web made of round shape spun filament (F) having a diameter of 10.5 μm, and made of homopolymer of polypropylene,
layer B is a pulp layer
layer C is a carded layer.

The spunbonded layer A was thermo-bonded with a calendar unit 2, using the aforesaid microbonding pattern C#1. This photography was taken on the spunbonded side (A) of the composite nonwoven. This composite nonwoven exhibits a high uniformity.

The invention is not limited to hydroentangled composite made of three layers (A/B/C), but the absorbent composite nonwoven could be made solely of the two layers A and B.

Furthermore, in case of a three layer composite nonwoven (A/B/C), the cover layer C is not necessarily a carded layer, but can be any other nonwoven layer, and in particular a spunbonded layer. For example, in the continuous system of FIG. 3, the carding unit 4 of FIG. 2 has been replaced by a second spunbonding unit 1′. In that case, the cover layer C of the composite nonwoven is not a carded layer, but is replaced by a spunbonded layer made of continuous filaments. The spunbonded layer C can be made of spun microfilaments having a diameter DI less or equal than 15 μm, or can be made of thicker spun filaments.

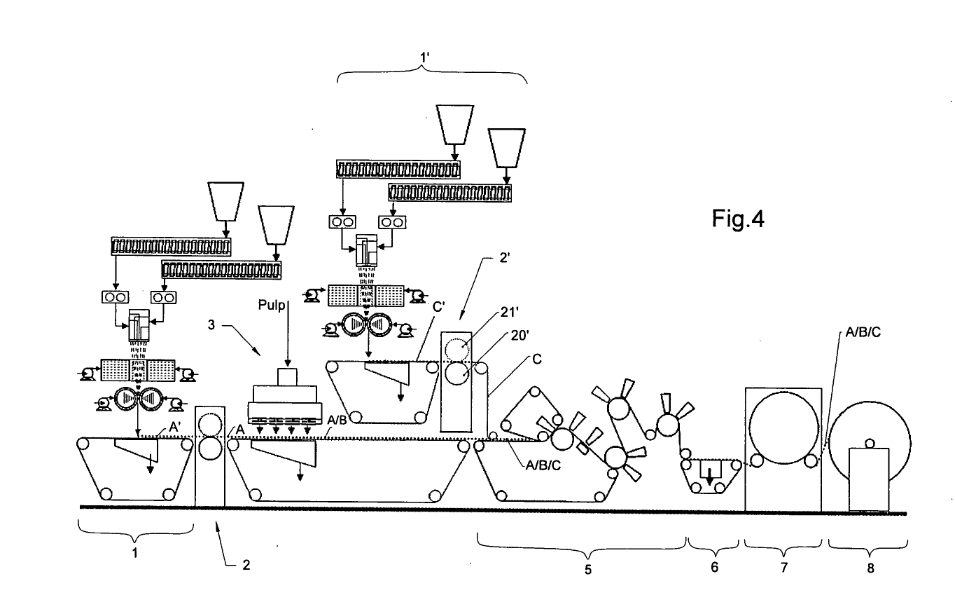

FIG. 4 shows another continuous system of the invention for producing a hydroentangled and absorbent composite nonwoven made of three layers. Compared with continuous system of FIG. 2, in the continuous system of FIG. 4, the carding unit 4 of FIG. 2 has been replaced by a second spunbonding unit 1′ and by a thermal bonding unit 2′ that are similar to the previously described spunbonding unit 1 and thermal bonding unit 2. In particular, the thermal bonding unit 2′ comprises two heated rolls 20′ and 21′. The lower roll 20′ has a smooth surface, and is for example a smooth steel roll. The upper roll 21′ has an engraved surface with protruding ribs, that are regularly distributed over the whole surface of the roll, and that form a bonding pattern having bonding dots 210a (like roll 21 of thermal bonding unit 2).

The continuous system of FIG. 4 is used for producing a hydroentangled and absorbent composite nonwoven made of three layers: a carrier layer constituted by the pre-consolidated spunbonded layer (A) previously described for the embodiment of FIG. 2; an intermediate pulp layer (B) previously described for the embodiment of FIG. 2; a cover layer (C) constituted by a pre-consolidated spunbonded layer.

In a preferred embodiment, the said second spunbonding unit 1′ is set up in order to produce a spunbonded web C′ made of spun filaments that comprise or are constituted by very fine spun microfilaments having a diameter DI less than or equal to 15 μm, and more preferably less than or equal to 10 μm. More preferably, the spunbonded web C′ is a web whose weight is between 7 g/m² and 35 g/m², preferably less than 25 g/m², more preferably less than 12 g/m². The density DD of the bonding dots 210a of the engraved roll 21′ is also very high, and at least equal to 90 bonding dots/cm², and more preferably at least equal to 100 bonding dots/cm²; the bonding ratio (R) is low, and preferably less than 30% and more preferably less than 20%. The area DA of each bonding dot 210a of engraved roll 21′ is less than 0.5 mm², preferably less than 0.3 mm², and more preferably less than 0.2 mm². Different shapes for bonding dots 210a of the engraved roll 21′ are suitable for practicing the invention (round shape, oval shape, square shape, rectangular shape . . . ). In this preferred embodiment, the microbonding pattern of the engraved roll 21′ can be the same as the microbonding pattern of the engraved roll 21 of thermal bonding unit 2, but this is not mandatory.

The composite nonwoven (A/B/C) produced with the aforesaid preferred embodiment of continuous system of FIG. 4 has advantageously comparable properties on both sides of the composite nonwoven.

The composite of the invention can comprise more than three superposed layers. For example, an additional layer (for example a carded layer) could be added underneath the spunbonded layer A′; in that case, this additional layer is for example laid onto conveyor belt 16, upstream the spunbonding unit 1, and the continuous spun filaments F are laid directly onto this additional layer.

The invention is not limited to the use of the pre-consolidated spunbonded web (A) of the invention for making the hydroentangled absorbent composite nonwovens (A/B) or (A/B/C) previously described in detailed in reference to the attached drawings. The pre-consolidated spunbonded web (A) of the invention can be used alone or can be used as a layer of any known types of composite nonwovens comprising at least two superposed layers. The composite nonwovens comprising the pre-consolidated spunbonded web (A) of the invention can be consolidated by any know bonding means, including notably thermal bonding, adhesive bonding, mechanical needling, hydrodynamic needling.

More especially, the pre-consolidated spunbonded web (A) of the invention can be used advantageously for making a hydroentangled spunbonded/carded nonwoven.

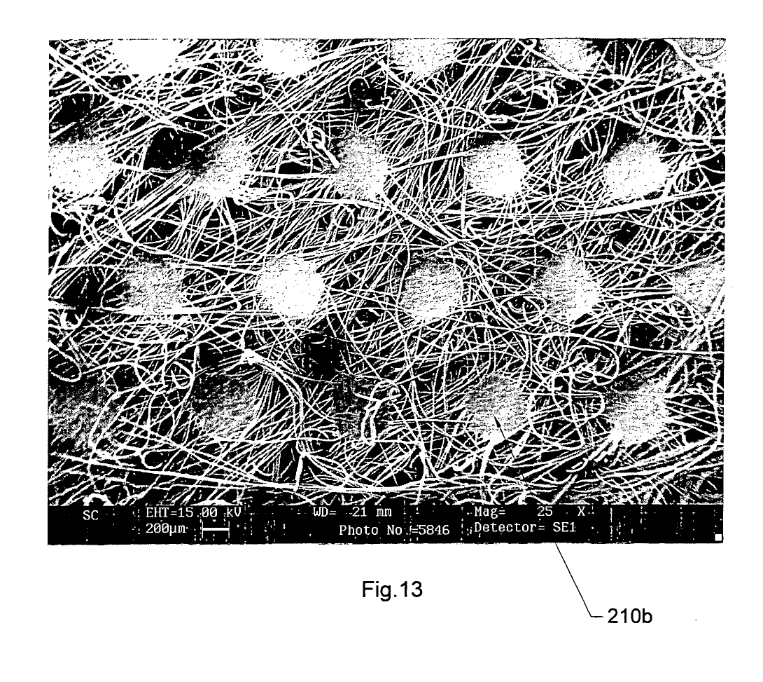

By way only of non-limiting example, FIG. 13 shows a photography of a sample of a hydroentangled composite nonwoven (A/C) of the invention, having a basis weight of 45 g/m², and wherein:
layer A is a pre-consolidated spunbonded web (15 g/m²) made of round shape spun filament (F) having a diameter of 10.5 μm, and made of homopolymer of polypropylene,
layer C is a carded layer (30 g/m²).

The spunbonded layer A was thermo-bonded with a calendar unit 2, using the aforesaid microbonding pattern C#1. This photography was taken on the spunbonded side (A) of the composite nonwoven.

Said hydroentangled spunbonded/carded nonwoven can be advantageously used for making the top sheets of diapers or training pants. Said hydroentangled spunbonded/carded nonwoven can be manufactured with a production line according to the one depicted on FIG. 2, but without the air-laying unit 3 or without using the air-laying unit 3.

1-59. (canceled)

60. A method of producing a pre-consolidated spunbonded web, said method comprising the steps of:
(a) forming one spunbonded layer,
(b) thermo-bonding the spunbonded layer, in order to obtain a pre-consolidated spunbonded web,
wherein the spunbonded layer formed in step (a) comprises continuous microfilaments having a diameter less than or equal to 15 μm, and wherein the pre-consolidation step (b) of the spunbonded layer is performed by means of a bonding pattern having bonding dots, the density of said bonding dots being higher than or equal to 90 dots/cm².

61. The method according to claim 60, wherein the pre-consolidation step (b) of the spunbonded layer is performed with a bonding pattern having a low bonding ratio less than 30%.

62. The method according to claim 60, wherein the pre-consolidation step (b) of the spunbonded layer is performed with a bonding pattern comprising bonding dots having a bonding area less than 0.5 mm².

63. The method according to claim 60, wherein the spunbonded layer comprises bilobal continuous filaments.

64. The method according to claim 60, wherein the weight of the spunbonded layer is not more than 15 g/m², and more preferably not more than 12 g/m².

65. The method of producing a hydroentangled composite nonwoven comprising at least two superposed layers, said method comprising the following steps:
forming one pre-consolidated spunbonded layer according to claim 60,
laying at least one second layer onto said pre-consolidated spunbonded layer,
consolidating the layers by hydrodynamic needling.

66. The method according to claim 65, wherein the second layer is one of a carded layer and a spunbonded layer.

67. The method according to claim 65 for producing a hydroentangled absorbent composite nonwoven, wherein the second layer is a pulp layer.

68. The method according to claim 65, further comprising an additional step of providing a nonwoven cover layer onto and in contact with the second layer before the step of consolidating the composite nonwoven by hydrodynamic needling.

69. The method according to claim 68, wherein the nonwoven cover layer is one of a carded layer and a spunbonded layer.

70. The method according to claim 69, wherein the additional step of providing a nonwoven cover layer comprises the following sub-steps:
(a′) forming one spunbonded layer
(b′) thermo-bonding the spunbonded layer, in order to obtain a pre-consolidated spunbonded layer.

71. The method according to claim 70, wherein the spunbonded layer formed in sub-step (a′) comprises continuous microfilaments having a diameter less than or equal to 15 μm, and wherein the pre-consolidation sub-step (b′) of the spunbonded layer is performed by means of a bonding pattern having bonding dots, the density of said bonding dots being higher than or equal to 90 dots/cm² for the bonding pattern used for making the pre-consolidated spunbonded layer.

72. The method according to claim 68, wherein the weight of the cover layer is less than 30 g/m².

73. The method according to claim 70, wherein the weight of the spunbonded layer is less than 35 g/m², preferably less than 25 g/m², more preferably less than 15 g/m², and even more preferably less than 12 g/m².

74. The method according to claim 67, wherein the weight of the pulp layer is less than 50 g/m².

75. The method according to claim 67, wherein the weight of the composite nonwoven is less than 115 g/m².

76. The method according to claim 68, wherein the weight of the composite nonwoven is between 27 g/m² and 115 g/m², wherein the weight of the pre-consolidated spunbonded carrier layer is between 7 g/m² and 35 g/m², wherein the weight of the pulp layer is between 10 g/m² and 50 g/m², and wherein the weight of the cover layer is between 10 g/m² and 30 g/m².

77. A pre-consolidated spunbonded web, comprising continuous microfilaments having a diameter less than or equal to 15 μm, and bonded dots, the density of said bonded dots being higher than or equal to 90 dots/cm².

78. The pre-consolidated spunbonded web according to claim 77, having a bonding ratio that is less than 30%.

79. The pre-consolidated spunbonded web according to claim 77, wherein the bonded dots have an area less than 0.5 mm².

80. The pre-consolidated spunbonded web according to claim 77, having a basis weight not more than 15 g/m², and preferably not more than 12 g/m².

81. The pre-consolidated spunbonded web according to claim 77, and comprising bilobal continuous filaments.

82. A composite nonwoven comprising at least one first and one second layers, wherein the first layer is a pre-consolidated spunbonded web including continuous microfilaments having a diameter less than or equal to 15 μm, and bonded dots, the density of said bonded dots being higher than or equal to 90 dots/cm².

83. The composite nonwoven according to claim 82, wherein the second layer is one of a carded layer, a pulp layer, and a spunbonded layer.

84. An absorbent hydroentangled composite nonwoven comprising a pre-consolidated spunbonded web including continuous microfilaments having a diameter less than or equal to 15 μm, and bonded dots, the density of said bonded dots being higher than or equal to 90 dots/cm² and an absorbent pulp layer in contact with the pre-consolidated spunbonded web.

85. The composite nonwoven according to claim 84, further comprising an additional nonwoven cover layer in contact with the absorbent pulp layer.

86. The composite nonwoven according to claim 85, wherein the nonwoven cover layer is one of a carded layer and a spunbonded layer.

87. The composite nonwoven according to claim 86, wherein the nonwoven cover layer is a pre-consolidated spunbonded web including continuous microfilaments having a diameter less than or equal to 15 μm, and bonded dots, the density of said bonded dots being higher than or equal to 90 dots/cm².

88. The composite nonwoven according to claim 84, wherein the weight of the cover layer is less than 30 g/m².

89. The composite nonwoven according to claim 84, wherein the weight of the pre-consolidated spunbonded web is less than 35 g/m², preferably less than 25 g/m², more preferably less than 15 g/m², and even more preferably less than 12 g/m².

90. The composite nonwoven according to claim 84, wherein the weight of the pulp layer is less than 50 g/m².

91. The composite nonwoven according to claim 84, wherein the weight of the composite nonwoven is less than 115 g/m².

92. The composite nonwoven according to claim 85, wherein the weight of the composite nonwoven is between 27 g/m² and 115 g/m², wherein the weight of the pre-consolidated spunbonded web is between 7 g/m² and 35 g/m², wherein the weight of the pulp layer is between 10 g/m² and 50 g/m², and wherein the weight of the cover layer is between 10 g/m² and 30 g/m².

93. Use of the composite nonwoven of claim 84 for making hygienic products, and more particularly one of dry wipes, wet wipes, diapers, training pants, sanitary napkins, and incontinence products.

94. A continuous system for producing a hydroentangled absorbent composite nonwoven according to claim 84, said system comprising:
a spunbonding unit for producing a spunbonded layer comprising continuous microfilaments having a diameter less than or equal to 15 μm,
a thermal bonding unit mounted downstream the spunbonding unit and comprising an engraved roll having a bonding pattern characterized by a bonding dots density that is higher than or equal to 90 dots/cm²,
an air-laying unit, mounted downstream the spunbonding unit, and fed with pulp,
a hydraulic needling unit mounted downstream the air-laying unit.

95. The continuous system according to claim 94, wherein the bonding ratio of the bonding pattern of engraved roll is less than 30%.

96. The continuous system according to claim 94, wherein the bonding pattern of engraved roll comprises bonding dots having an area less than 0.5 mm².

97. The continuous system according to claim 94, further comprising a carding unit upstream the hydraulic needling unit.

98. The continuous system according to claim 94, further comprising an additional spunbonding unit upstream the hydraulic needling unit.

99. The continuous system according to claim 98, wherein the additional spunbonding unit is set up in order to produce a spunbonded layer comprising continuous microfilaments having a diameter less than or equal to 15 μm, and preferably less than or equal to 10 μm.

100. The continuous system according to claim 99, comprising an additional thermal bonding unit that is mounted downstream the additional spunbonding unit and that comprises an engraved roll, and wherein said engraved roll has a bonding pattern characterized by a bonding dots density that is higher than or equal to 90 dots/cm² for the engraved roll of the other thermal bonding unit.
